(19) United States
(12) Patent Application Publication
(10) Pub. No.: US 2002/0107536 A1
(43) Pub. Date: Aug. 8, 2002

(54) Device and method for preventing kidney failure

(76) Inventor: Hany M. Hussein, Newport Coast, CA (US)

Correspondence Address: JAMES G O'NEILL, 3151 AIRWAY AVE SUITE K105, COSTA MESA, CA, 926264613

(21) Appl. No.: 10/071,418

(22) Filed: Feb. 7, 2002

Related U.S. Application Data
(60) Provisional application No. 60/267,255, filed on Feb. 7, 2001.

(51) Int. Cl. A61B 17/08

(52) U.S. Cl. 606/158

(57) ABSTRACT

A method and devices for protecting kidneys from irreversible damage. The method is based on controlling or restricting blood flow through renal arteries in order to prevent kidney failure. The devices are delivered to a renal artery for restricting the flow of blood through the artery to the kidney. The devices may take the form of internal restrictors, such as an orifice, a venturi, a tapered coil, or a perforated sleeve that is inserted in a renal artery, or external devices such as an inflatable cuff or balloon, or a clamp that is placed around a renal artery. The improved method of the present invention includes means for delivering the internal devices to renal arteries by a percutaneous catheter system, or external devices by endoscopic devices or surgery.

### CROSS REFERENCE TO RELATED APPLICATION

This application is a continuation of pending provisional application No. 60/267,255 filed on Feb. 7, 2001.

### FIELD OF THE INVENTION

This invention relates generally to medical devices and methods, and more particularly, to an improved device for and method of preventing kidney failure.

### DESCRIPTION OF RELATED ART

Clinical observation indicates that diabetic kidneys that receive their blood supply from stenosed renal arteries tend to be spared from renal failure compared to kidneys that receive their blood supply from patent renal arteries. Kidney dialysis and kidney transplants are costly and traumatic alternatives for treatment of kidney failure. Therefore, there exists a need in the art for an easy-to-use, less invasive and less costly device and method for protecting kidneys from irreversible damage that leads to renal failure.

### SUMMARY OF THE INVENTION

Accordingly, it is a general object of the present invention to provide an improved device to prevent renal failure. It is a particular object of the present invention to provide an improved device and method for preventing kidney failure. It is a still more particular object of the present invention to provide an improved device and method that restricts blood flow through the renal artery. It is yet a further particular object of the present invention to provide an improved external or internal device such as a flow restrictor or clamping means for controlling blood flow through the renal artery. And, it is particular object of the present invention to provide an improved method for preventing kidney failure by inserting devices that restrict blood flow through the renal artery, externally around, or internally into the renal artery.

In accordance with one aspect of the present invention, there are provided blood flow restriction devices for protecting kidneys from irreversible damage, which devices are delivered to a renal artery for restricting the flow of blood through the artery to the kidney. The devices may take the form of internal restrictors that are inserted in a renal artery, or external devices that are placed around a renal artery. The improved method of the present invention includes means for delivering internal devices to renal arteries by a percutaneous catheter system, or external devices by endoscopic means or surgery.

### BRIEF DESCRIPTION OF THE DRAWINGS

The objects and features of the present invention, which are believed to be novel, are set forth with particularity in the appended claims. The present invention, both as to organization and manner of operation, together with further objects and advantages, may best be understood by reference to the following description, taken in conjunction with the accompanying drawings, in which:

FIG. 1 is a cross-sectional view of a preferred embodiment of an internal flow restricting device of the present invention, inserted in a renal artery;

FIG. 2 is a cross-sectional view of the preferred embodiment of the internal flow restricting device of FIG. 1 removed from the renal artery;

FIG. 3 is a cross-sectional view of a further embodiment of an internal flow restricting device, in the form of a tapered coil;

FIG. 4 is a cross-sectional of a further embodiment of an internal flow restricting device, in the form of a perforated sleeve made from foil, braid or mesh;

FIGS. 4A and 4B are side elevational views of further embodiments of internal flow restricting devices, in the form of perforated sleeves made from foil, braid or mesh;

FIG. 5 is a cross-sectional view of a first embodiment of an external flow restricting device, in the form of an inflatable cuff or balloon, held around a renal artery; and

FIG. 6 is a cross-sectional view of a further embodiment of an external flow restricting device, in the form of a clamp, held around a renal artery.

### DESCRIPTION OF THE PREFERRED EMBODIMENTS

The following description is provided to enable any person skilled in the art to be able to use the invention and sets forth the best modes contemplated by the inventor for carrying out his invention. Various modifications, however, will remain readily apparent to those skilled in the art, since the generic principals of the present invention have been defined herein, specifically to provide for an improved device and method for protecting kidneys from irreversible damage that leads to renal failure, especially among diabetics.

The devices of the present invention are delivered to or inserted in or around a renal artery by a percutaneous catheter system (for internal devices), or by endoscopic means or surgery (for external devices).

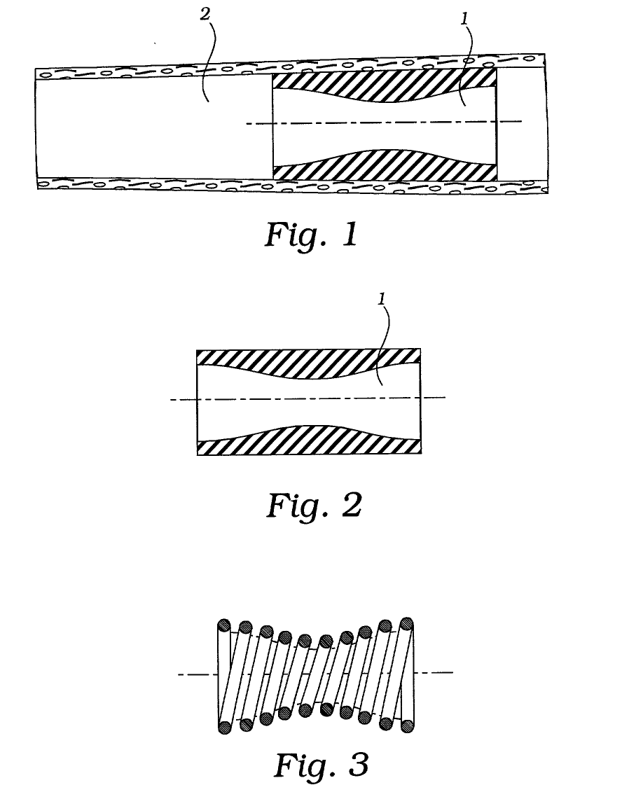

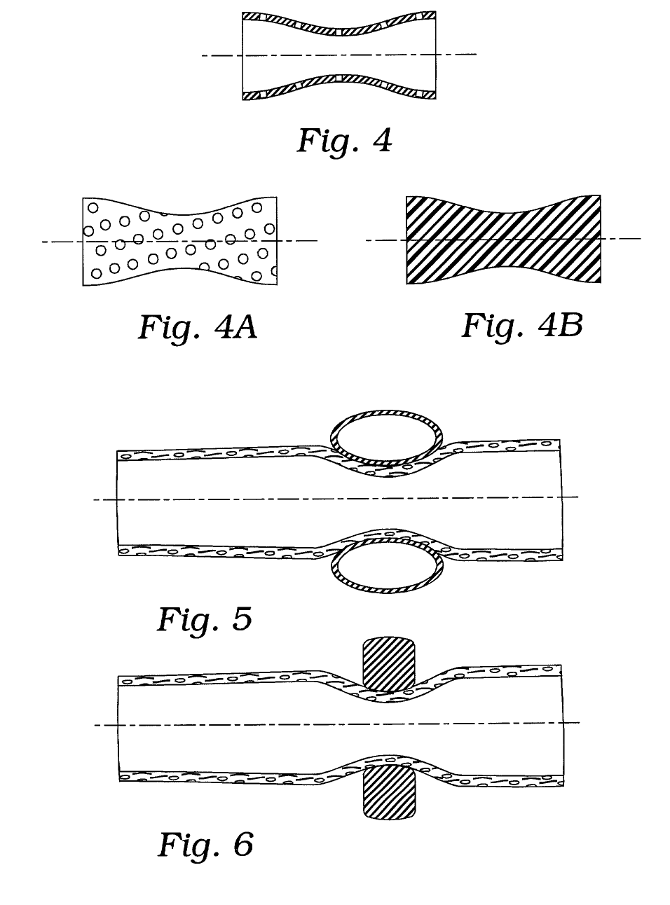

As best shown in FIGS. 1-4B, internal devices of the present invention include a housing having internal flow restricting means 1, 3 or 4, such as an orifice or venturi formed internally in a body made in the form of a sleeve, a tapered coil or a perforated sleeve made from a foil, braided or mesh material. These internal devices are inserted into the internal passage 2 of a renal artery to restrict flow of blood through the artery.

As best shown in FIGS. 5 and 6, external devices of the present invention may take the form of an inflatable cuff or balloon 6 or a clamp 7 that is held around a renal artery to from a restriction 5 in the artery, to control the flow of blood therethrough.

It, therefore, can be seen that the present invention provides a novel and improved device and method for protecting kidneys from irreversible damage that leads to renal failure, by controlling the flow of blood through the renal artery.

Those skilled in the art will appreciate that various adaptations and modifications of the just-described, preferred embodiments can be configured without departing from the scope and spirit of the invention. Therefore, it is to be understood that, within the scope of the appended claims, the invention may be practiced other than as specifically described herein.

What is claimed is:

1. A device for preventing kidney failure, comprising:
a housing having an inside surface, an outside surface and two ends;
an internal passage extending between the two ends and bound by the inside surface; and
the device being adapted to be inserted in or around a renal artery to restrict blood flow through a renal artery.

2. The device of claim 1 wherein the device includes an internal flow restricting means to restrict blood flow.

3. The device of claim 2 wherein the housing is a sleeve.

4. The device of claim 3 wherein the sleeve is perforated.

5. The device of claim 4 wherein the perforated sleeve is made from a foil material.

6. The device of claim 4 wherein the perforated sleeve is made from a braided material.

7. The device of claim 4 wherein the perforated sleeve is made from a mesh material.

8. The device of claim 2 wherein the housing is a tapered coil.

9. The device of claim 1 wherein the housing is an inflatable cuff for placement around an outside surface of a renal artery.

10. The device of claim 1 wherein the housing is a clamp for placement around an outside surface of a renal artery.

11. A method of preventing kidney failure comprising the steps of:
forming a device for use with a renal artery to restrict blood flow through the renal artery; and
inserting the device in or on the renal artery to restrict blood flow through the renal artery.

12. The method of claim 11 wherein the device is a flow constrictor having an internal flow restricting means.

13. The method of claim 12, including the step of inserting the flow constrictor in the renal artery by means a percutaneous catheter system.

14. The method of claim 11 wherein the device is inserted over an outside surface of the renal artery.

15. The method of claim 14 wherein the device is inserted over the outside surface of the renal artery by endoscopic means.

16. The method of claim 15 wherein the device is an inflatable cuff.

17. The method of claim 15 wherein the device is a clamp.

18. A device for preventing kidney failure by insertion into a renal artery, comprising:
a housing having an inside surface, an outside surface and two ends; and
an internal passage extending between the two ends and bound by the inside surface; a flow restrictor for restricting blood flow through the renal artery formed in the internal passage.

19. The device of claim 18 whreein the housing is in the form of a sleeve or a tapered coil.

20. The device of claim 19 wherein the sleeve is perforated and made from a material selected from the group of foil, braid or mesh.

21. A device for preventing kidney failure by placement around an outside surface of a renal artery, comprising:
a housing having an inside surface, an outside surface and two ends; and
the housing has a restricted internal passage between the two ends to restrict blood flow through the renal artery.

22. The device of claim 21 wherein the device is an inflatable cuff.

23. The device of claim 22 wherein the device is a clamp.
